(19) United States
(12) Patent Application Publication
(10) Pub. No.: US 2014/0100438 A1
(43) Pub. Date: Apr. 10, 2014

(54) OBJECT INFORMATION ACQUIRING APPARATUS AND CONTROL METHOD FOR SAME

(71) Applicant: CANON KABUSHIKI KAISHA, Tokyo (JP)

(72) Inventor: Yoshiko Wada, Kyoto-shi (JP)

(73) Assignee: CANON KABUSHIKI KAISHA, Tokyo (JP)

(21) Appl. No.: 14/029,010

(22) Filed: Sep. 17, 2013

(30) Foreign Application Priority Data
Oct. 10, 2012 (JP) 2012-224945

(51) Int. Cl. A61B 5/00

(52) U.S. Cl. CPC A61B 5/0095; A61B 5/7246
USPC 600/407

(57) ABSTRACT

An object information acquiring apparatus, including: a probe converting an acoustic wave generated from an object irradiated with light into a detection signal; and a signal processing unit generating information about the object from the detection signal, wherein the signal processing unit: extracts a sampling signal on the basis of conditions relating to the signal shape; generates a reference signal from the sampling signal; detects a phase-inverted signal, which is a portion of the detection signal where the phase is inverted with respect to the reference signal, by carrying out a correlation calculation; generates a signal after suppression by suppressing the intensity of the phase-inverted signal from the detection signal; and generates information about the interior of the object using the signal after suppression.

### BACKGROUND OF THE INVENTION

1. Field of the Invention

The present invention relates to an object information acquiring apparatus and a control method for same.

2. Description of the Related Art

In the field of medicine, there has been active research into optical imaging apparatuses which irradiate light onto an object under inspection, such as a living organism, from a light source, such as a laser, and create an image of information on the interior of the object obtained on the basis of the incident light. One example of optical imaging technology of this kind is photoacoustic imaging (PAI). In photoacoustic imaging, pulsed light generated by a light source is irradiated onto an object, an acoustic wave (typically, an ultrasonic wave) generated by the tissue of the object that has absorbed the energy of the pulsed light which has propagated and diffused inside the object is detected, and object information is imaged (formed into an image) on the basis of this detection signal.

In other words, using the difference in absorptivity of the light energy in an examination segment, such as a tumor, and other tissue, the elastic wave (photoacoustic wave) generated when the examination segment momentarily expands upon absorbing irradiated light energy is received with a probe. By mathematically analyzing this detection signal, it is possible to obtain an optical characteristics distribution inside the object, and in particular, an initial sound pressure distribution, a light energy absorption density distribution or an absorption coefficient distribution, etc.

This information can be used for quantitative measurement of specific properties inside the object, such as the oxygen saturation inside the blood, for example. In recent years, pre-clinical research which uses photoacoustic imaging of this kind to create images of blood vessels of small animals, and clinical research which applies this principle to the diagnosis of breast cancer, and the like, has been pursued actively (see “Photoacoustic imaging in biomedicine”, M. Xu, L. V. Wang, Review of Scientific Instruments, 77, 041101, 2006). In photoacoustic imaging, normally, the aim is to create an image of the optical characteristics distribution of a light absorbing body, which is inside the object.
Non Patent Literature 1: “Photoacoustic imaging in biomedicine”, M. Xu, L. V. Wang, Review of Scientific Instruments, 77, 041101, 2006

### SUMMARY OF THE INVENTION

However, due to the presence of members which hold the object, and the like, and which are situation to the exterior of the living organism, the photoacoustic wave is multiply reflected and the multiply reflected photoacoustic wave is also received by the probe. If an optical characteristics distribution is created using a detection signal which includes a reflected wave, then there is a problem in that an artifact due to the reflected wave is produced and image deterioration occurs.

The present invention was devised in view of the aforementioned circumstances, an object thereof being to provide an object information acquiring apparatus which is capable of reducing image deterioration caused by the effects of reflected waves that may be detected.

The present invention provides an object information acquiring apparatus, comprising:

a probe configured to convert an acoustic wave generated from an object irradiated with light into a detection signal; and

a signal processing unit configured to generate information about the interior of the object from the detection signal, wherein

the signal processing unit is configured to:

extract a sampling signal on the basis of signal sampling conditions, which are conditions relating to the signal shape, from the detection signal;

generate a reference signal from the sampling signal;

detect a phase-inverted signal, which is a portion of the detection signal where the phase is inverted with respect to the reference signal, by carrying out a correlation calculation of the detection signal and the reference signal;

generate a signal after suppression by suppressing the intensity of the phase-inverted signal from the detection signal; and

generate information about the interior of the object using the signal after suppression.

The present invention also provides a control method for an object information acquiring apparatus including: a probe converting an acoustic wave generated from an object irradiated with light into a detection signal; and a signal processing unit generating information about the interior of the object from the detection signal,

the method comprising, by means of the signal processing unit:

extracting a sampling signal on the basis of signal sampling conditions, which are conditions relating to the signal shape, from the detection signal;

generating a reference signal from the sampling signal;

detecting a phase-inverted signal, which is a portion of the detection signal where the phase is inverted with respect to the reference signal, by carrying out a correlation calculation of the detection signal and the reference signal;

generating a signal after suppression by suppressing the intensity of the phase-inverted signal from the detection signal; and

generating information about the interior of the object using the signal after suppression.

According to the present invention, it is possible to provide an object information acquiring apparatus which can reduce image deterioration caused by the effects of detecting reflected waves.

Further features of the present invention will become apparent from the following description of exemplary embodiments with reference to the attached drawings.

### BRIEF DESCRIPTION OF THE DRAWINGS

FIG. 1 is a schematic drawing showing a composition of a photoacoustic image-forming apparatus;

FIG. 2 is a schematic drawing showing a composition of a signal processing unit of a photoacoustic image-forming apparatus;

FIG. 3 is a conceptual diagram illustrating a process of the occurrence of phase inversion when an acoustic wave is reflected;

FIG. 4 is a conceptual diagram showing one example of suppression of a reflected signal in a signal processing unit of a photoacoustic image-forming apparatus;

FIG. 5 is a diagram showing a signal before and after suppression of a reflected wave;

FIGS. 6A and 6B are diagrams showing an image of the interior of an object before and after suppression of a reflected wave; and

FIG. 7 is a flowchart showing processing in a signal processing unit.

### DESCRIPTION OF THE EMBODIMENTS

A desirable mode for carrying out the present invention is described below with reference to the drawings. The dimensions, materials, shapes and relative positions, and the like, of the constituent parts described below should be changed appropriately depending on the composition and various conditions of the apparatus to which the invention is applied, and it is not intended to limit the scope of the invention to the description given below.

The object information acquiring apparatus according to the present invention includes an apparatus which receives an acoustic wave generated inside an object by irradiating light (an electromagnetic wave) and which uses a photoacoustic effect to acquire the object information as image data. The acquired object information means the generating source distribution of the acoustic waves which have been generated by light irradiation, the initial sound pressure inside the object, or the light energy absorption density distribution or absorption coefficient distribution, and the density distribution of material constituting the tissue, which are derived from the initial sound pressure distribution. The material density distribution is, for example, the oxygen saturation distribution, the oxidized/reduced hemoglobin density distribution, and the like.

The acoustic wave referred to in the present invention is typically an ultrasonic wave, and includes elastic waves called sound waves, ultrasonic waves and acoustic waves. An acoustic wave generated by the photoacoustic effect is called a photoacoustic wave or a photoultrasonic wave. The probe receives an acoustic wave generated or reflected inside the object.

In the description given below, a photoacoustic image-forming apparatus which generates an image on the basis of the acquired object information is described by way of an example. However, the mode of implementing the present invention is not limited to this and it is not particularly necessary to form an image, but rather, the objection information may be saved or output as data.

### Basic Embodiment

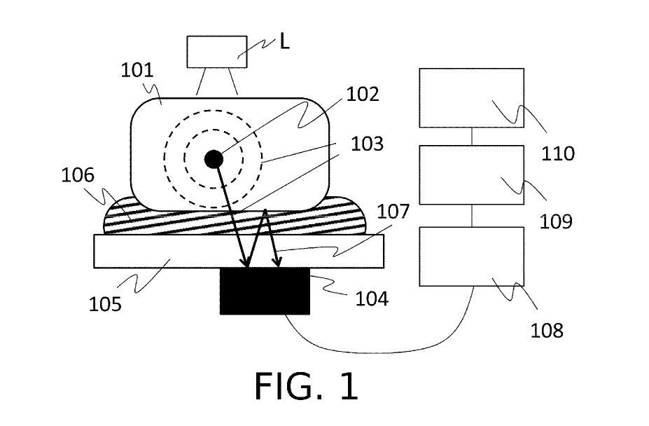

The composition of a photoacoustic image-forming apparatus relating to the present embodiment is described here with reference to FIG. 1.

This photoacoustic image-forming apparatus comprises, as a basic structure: a light source L, a holding member 105, a probe 104 having a plurality of elements which receive acoustic waves, and a signal processing unit 109. When the pulsed light (electromagnetic wave) output from a light source is irradiated onto the object 101, it may pass via optical components, such as lenses, mirrors, or the like.

An absorbing body 102 inside the object absorbs the irradiated light and generates an acoustic wave 103. The acoustic wave 103 propagates inside the living organism, passes through an acoustic matching material 106 and the holding member 105, and is detected by the probe 104. Furthermore, a portion of the acoustic wave is reflected by the boundary surface of the holding member 105, and the like, to form a reflected wave 107. The probe 104 also detects the reflected wave 107.

A front-stage processing unit 108 carries out amplification and digital conversion on the acoustic wave (and reflected wave) which have been detected and converted into an electrical signal by the probe 104. The signal processing unit 109 applies image reconstruction processing to the signal after digital conversion, thereby converting the object information into image data, which is displayed on the display unit 110. The signal processing unit 109 according to the present invention is characterized in that processing is carried out to reduce image artifacts caused by multiple reflection at the holding member 105 and/or the acoustic matching material 106.

(Phase Inversion of Acoustic Wave Due to Reflection)

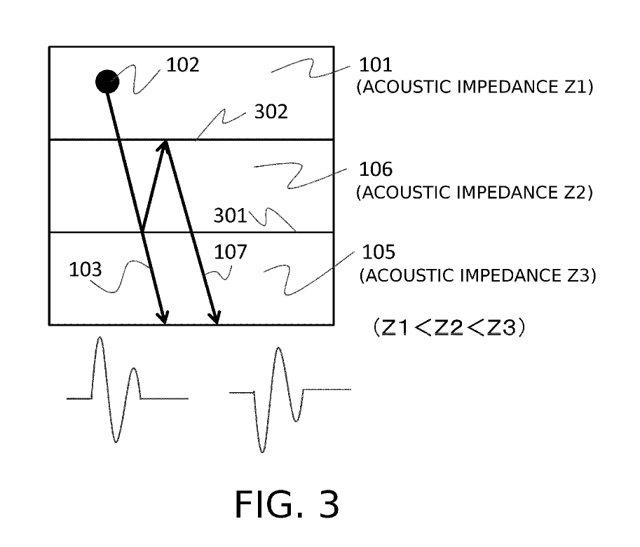

The physical phenomenon of an acoustic wave of which the phase of the reflected component is inverted through 180° will now be described with reference to FIG. 3. The material through which the acoustic wave propagates has an acoustic impedance value that indicates the ease with which the acoustic wave can pass through the material. Here, when the acoustic wave which has propagated through a particular material is incident on a material having a different acoustic impedance value, then reflection or diffraction occurs. It is known that in the reflected wave of an acoustic wave which is incident on a material having a higher acoustic impedance value, from a material having a lower acoustic impedance value, the phase is inverted through 180°.

For example, as shown in FIG. 3, a case can be imagined in which the acoustic wave 103 generated by the absorbing body 102 propagates through the object 101, the acoustic matching material 106 and the holding member 105. The acoustic wave is reflected due to the occurrence of a change in the acoustic impedance, at the boundary surface 301 between the acoustic matching material 106 and the holding member 105, and at the boundary surface 302 between the object 101 and the acoustic matching material 106.

The acoustic impedance of the holding member 105 is termed Z3, the acoustic impedance of the acoustic matching material 106 is termed Z2, and the acoustic impedance of the object is termed Z1. In general, the acoustic impedance of the holding member is higher than that of the object (Z3>Z1). Furthermore, since the acoustic matching impedance is inserted in order to achieve acoustic matching between the object and the holding member, then the acoustic impedance values have the relationship: Z3>Z2>Z1. Therefore, when the acoustic wave is incident on the holding member 105 from the acoustic matching material 106, the phase of the reflected wave 107 which is reflected by the boundary surface 301 is inverted through 180° (since Z2<Z3).

Thereupon, the reflected wave is also reflected at the boundary surface 302 between the object 101 and the holding member 105. In this case, since the reflected wave is incident on a material of lower acoustic impedance from a material of higher acoustic impedance (since Z2>Z1), then inversion of the phase does not occur. In this way, the reflected wave 107 which is incident on the probe is a signal having a phase that is 180 degrees different from the phase of the acoustic wave 103 from the absorbing body. Since this reflected wave 107 has been reflected, the acquisition time thereof is delayed with respect to the acoustic wave 103, and the reflected wave has characteristics in which the phase is inverted through 180°.

(Signal Processing Unit 109)

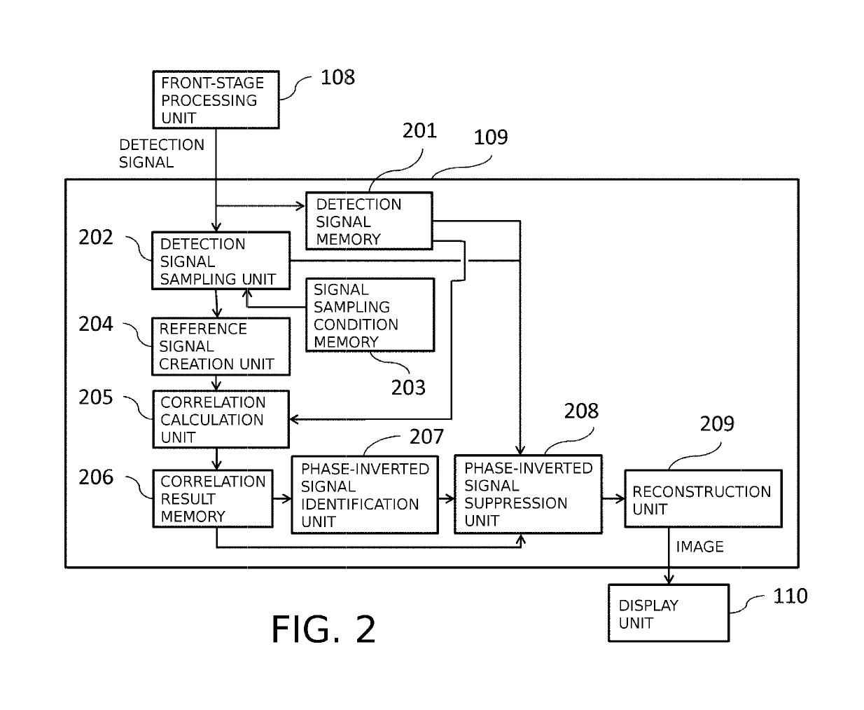

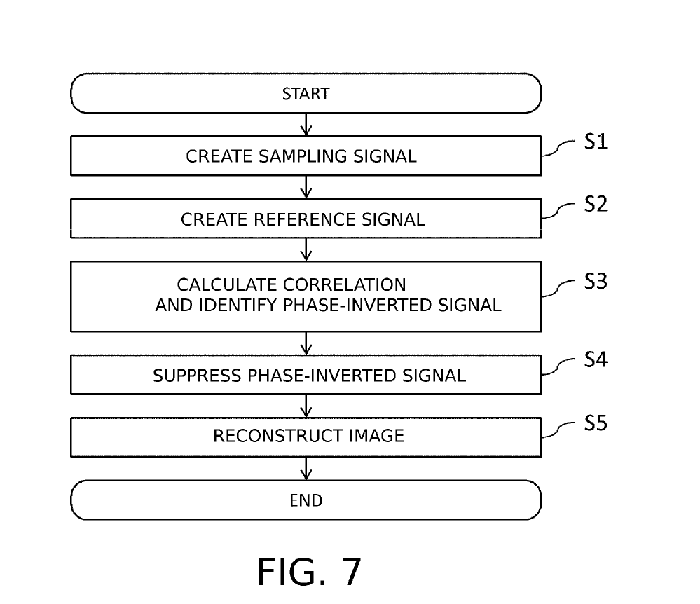

The internal composition of the signal processing unit 109 which is the characteristic feature of the present invention is described here with reference to FIG. 2. At the same time as this, specific processing will be described with reference to the flowchart in FIG. 7.

<Step S1: Creation of Sampling Signal>

When the signal processing unit 109 receives the digitalized detection signal from the front-stage processing unit 108, firstly, a detection signal sampling unit 202 samples a portion of each detection signal on the basis of the conditions recorded in the signal sampling conditions memory 203, to form a sampling signal. Furthermore, simultaneously, the detection signal is stored in the detection signal memory 201.

The signal sampling conditions may relate to the position, width and shape of the sampling signal.

The position of the sampling signal (the appearance time of the signal after the irradiation of light) is decided by the position of the absorbing body under investigation. The sampling signal position can be calculated by using the distance from the probe to the absorbing body, and the speed of sound in the portion through which the acoustic wave passes. If the position of the absorbing body is not decided by calculation, then the position of the absorbing body is determined by confirming the characteristics of the detection signal.

The signal width is desirably a width equivalent to the probe response signal. If the sampling signal width is not suitable, then erroneous judgments are frequent in the phase inversion signal identification in S3.

The signal shape depends on the size and form of the absorbing body under investigation. Typically, the signal shape which is generated from a spherical absorbing body is set as the signal sampling conditions, and similar shapes are detected. For example, when extracting the sampling signal from the detection signal, a correlation is found between the detection signal and the set shape, and a signal having a high correlation with the set shape is extracted.

The signal shape is also dependent on the response characteristics of the probe. Depending on the response characteristics of the probe, there are restrictions on the frequency band of the received photoacoustic wave, and the shape of the detection signal changes. By compensating for this shape change, it is possible to achieve highly accurate processing. The signal shape is also dependent on the attenuation of the photoacoustic signal. In other words, since the attenuation coefficient becomes greater in direct proportion to the frequency of the photoacoustic wave, then the frequency component of the photoacoustic wave is changed and the signal shape is changed. Therefore, by setting the response function of the probe and the frequency attenuation due to propagation to the signal sampling conditions, sampling processing of higher accuracy becomes possible.

If there is a plurality of original detection signals, then it is possible to sample a plurality of sampling signals. In cases of this kind, it is possible to create a plurality of sampling signals corresponding to each detection signal, or to sample one signal from any of the detection signals. Moreover, it is also possible to select a plurality of detection signals from all of the detection signals and to set this as a sampling signal. The signal sampling conditions may be changed in accordance with the detection signal, or universal conditions may be used.

<Step S2: Creation of Reference Signal>

The reference signal creation unit 204 creates a reference signal on the basis of the sampling signal created in S1.

The reference signal is used as a reference signal when calculation the correlation in a subsequent stage. In order to improve the accuracy of identification of the reflected signal, it is desirable to create a reference signal by predicting the shape of the reflected signal.

Now, an example of a method of creating a reference signal will be given. Firstly, there is a method which simply takes the sampling signal as a reference signal.

Furthermore, if the SN ratio of the sampling signal is poor, then in order to improve the SN ratio, there is also a method which takes the average of a plurality of sampling signals as a reference signal. It is also possible to utilize a weighted average in accordance with various criteria, rather than a simple average.

Moreover, it is also possible to envisage a method which takes account of the change in the shape of the reflected signal due to frequency attenuation when the signal propagates through a longer distance than the sampling signal, and which compensates by adjusting the frequency component of the sampling signal in accordance with the amount of attenuation, to create a reference signal. In this case, it is also possible to create a plurality of reference signals in accordance with the extent of the attenuation. Also, there is a method which extends the sampling signal in the time axis direction, and a method which changes the amplitude by a function of time.

<Step S3: Calculation of Correlation and Identification Of Phase Inversion Signal>

The correlation calculation unit 205 calculates the correlation between the respective detection signals and the reference signal, and stores the results in a correlation result memory 206. Subsequently, from these calculation results, the phase inversion signal identification unit 207 identifies a signal portion (phase-inverted portion) in which the phase differs by 180 degrees from the reference signal.

The correlation calculation unit employs a time window function for the length of the reference signal, to the sampling signal, and calculates the correlation between the reference signal and the detection signal, while shifting the window function in the time direction. In this case, it is desirable to use a reference signal of which the frequency component has been adjusted in accordance with the time period for which the correlation calculation is carried out, or to carry out a correlation calculation while adjusting the frequency component of the reference signal. The reason for this is that, since frequency attenuation occurs in the reflected signal that has propagated through a long distance, then the frequency component changes in direct proportion to an index function of the propagation distance.

The phase inverted signal identification unit identifies the signal portion having a reflected component of the reference signal, from the correlation results. The correlation result shows a large positive value when the detection signal has a shape similar to the reference signal, and shows a large negative value when the detection signal has a phase which is inverted by 180 degrees. Therefore, a signal having a high negative correlation is identified as an inverted component of the reference signal. By setting the reception time range of the reflection signal, it is possible to select the identified signal range. By setting a plurality of signal identification ranges, it is possible to identify a plurality of reflection positions. The intensity of this reflection signal is determined by the intensity of the reference signal and the reflectivity at the reflection surface. If the reflectivity is known, and the intensity of the reflected signal can be predicted, then it is desirable to add the intensity of the reflected signal as a specific condition.

<Step S4: Suppression of Phase-Inverted Signal>

The phase-inverted signal suppression unit 208 generates a signal after suppression by applying correction so as to suppress the effects of phase inversion, to the phase-inverted portion identified in S3.

As a method for suppressing the reflection signal, firstly, there is a method which sets the signal amplitude of the phase-inverted portion to zero. In this case, the average of the phase-inverted signal portion identified in S3 is substituted for this portion. Furthermore, the intensity may also be set to an arbitrary suitable value, rather than zero.

As a further suppression method, there is also a method for suppressing the amplitude by creating a suppression signal using the reference signal and subtracting this signal so as to align the intensity of the suppression signal with the intensity of the phase-inverted signal. A method which aligns the intensities of the phase-inverted signal and the suppression signal may be a method which aligns the maximum intensities of both signals, or a method which applies a suitable magnification gain to the amplitude of the suppression signal, so as to minimize the difference between the intensities. If the frequency attenuation of the reflection component is not to be taken into account in the reference signal, then it is possible to create the reference signal by adjusting the frequency of the suppression signal when the suppression signal is created. If the phase-inverted signal is suppressed by using a suppression signal, then it is possible to leave the signal apart from the reflection component.

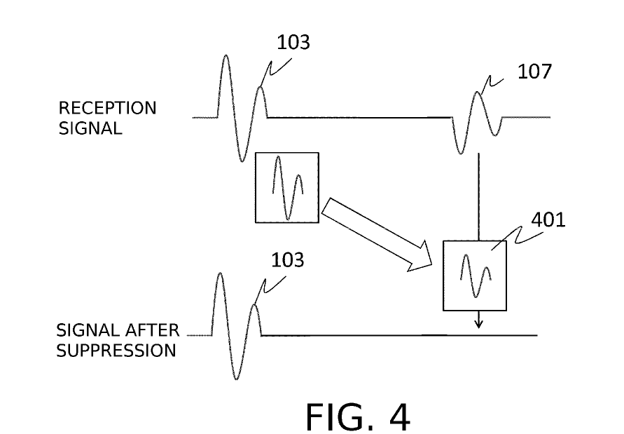

FIG. 4 shows the aspect of the suppression of the inverted signal. The signal in the upper portion of FIG. 4 is the original reception signal, which includes a component corresponding to the reflected wave 107. This reflection component has a phase that is inverted compared to the acoustic wave 103 of the reception signal. Therefore, in this example, a suppression signal 401 is created from a reference signal obtained by directly using the sampling signal, so as to be aligned with the intensity of the phase-inverted signal, and this suppression signal is subtracted from the phase-inverted signal. By this means, a signal after suppression such as that shown in the lower part of FIG. 4 is generated.

<Step S5: Image Reconstruction>

The reconstruction unit 209 generates image data representing the object information, by carrying out image reconstruction using the signal after suppression. The generated image data is sent to the display unit 110 and displayed.

The image reconstruction method used is, for example, back projection in a time domain or Fourier domain, as commonly used in tomography technology. The output image is displayed on the display unit 110.

In this flowchart, the sampling signal created in S1 includes a photoacoustic signal which is generated from the absorbing body (a digital signal originating in a photoacoustic wave). Furthermore, the phase-inverted signal identified in S3 includes a reflected signal which is the cause of an artifact (a digital signal originating in the reflected wave). Therefore, by suppressing this phase-inverted signal, it is possible to suppress the reflected signal component in the photoacoustic signal. The signal processing unit according to the present invention is able to suppress the reflected component of the acoustic wave generated by the absorbing body, by setting the signal sampling conditions in accordance with the position and shape of the absorbing body that is under investigation.

In the description given above, suppression of a reflected signal at the boundary surface 301 between the holding member 105 and the acoustic matching material 106 is described by way of an example, but a reflected wave having a similarly inverted phase is generated when the acoustic impedance is increased or decreased in steps. In a photoacoustic image-forming apparatus having a composition of this kind, the processing according to the present invention is effective.

In the present description, the suppression of the reflection component is carried out before reconstruction, but suppression may also be carried out after creating an image. In this case, the signal is input to the reconstruction unit of the signal processing unit, and then output to the detection signal sampling unit, and the processing steps are carried out in the order: S5, S1, S2, S3, S4.

Below, the main constituent elements of the apparatus will be described.

(Light Source L)

The light source irradiates light onto the object. When light of a prescribed wavelength from the light source is absorbed by the absorbing body, which is a component inside the object that corresponds to the wavelength of the light, then a photoacoustic wave is generated. For the light source, it is desirable to use a pulsed light source capable of generating, as irradiation light, pulsed light of the order of several nanosecond to several hundred nanosecond. More specifically, in order to generate a photoacoustic wave efficiently, a pulse width of approximately 10 nanoseconds is used.

For the light source, it is desirable to employ a laser since a large output can be obtained, but it is also possible to use a light-emitting diode, or the like, instead of a laser. As a laser, it is possible to use lasers of various types, such as a solid-state laser, a gas laser, a fiber laser, a color laser, a semiconductor laser, or the like. The irradiation timing, waveform, intensity, and the like, are controlled by a light source control unit, which is not illustrated.

In the present invention, the wavelength of the light source used is desirably a wavelength at which the light propagates to the interior of the object when the object is a living body. More specifically, the wavelength is no less than 500 nm and no more than 1200 nm.

(Optical System)

The optical system (not illustrated) shapes the light irradiated from the light source into a desired light distribution shape, and then guides the light to the object. For the optical system, it is possible to use mirrors which reflect light, lenses which condense or enlarge the light and change the shape of the light, optical components, such as a diffusion plate which diffuses light, a light waveguide, such as an optical fiber, or the like. Rather than condensed the light by, for example, the lens, the light may be spread to a certain surface area, from the viewpoint of improving safety and increasing the diagnosis area.

(Probe 104)

The probe may also be called a transducer, and detects an acoustic wave and converts it into an analog electrical signal. It is possible to use an acoustic wave probe of any kind, such as a probe based on a piezoelectric effect, a probe based on light resonance, or a probe based on change in capacitance, or the like, provided that the probe is capable of detecting an acoustic wave signal. In the probe, typically, a plurality of light-receiving elements may be arranged in a one-dimensional or a two-dimensional configuration. By using a multi-dimensional arrangement of elements in this way, it is possible to detect acoustic waves in a plurality of locations, simultaneously, and hence the detection time can be shortened and the effects of vibration of the object, and the like, can be reduced.

(Front-Stage Processing Unit 108)

The front-stage processing unit amplifies and digitally converts the analog electrical signal. The front-stage processing unit is typically constituted by an amplifier, an A/D converter, a field programmable gate array (FPGA) chip, and the like. Desirably, if a plurality of detection signals are obtained from the probe, then the front-stage processing unit is able to process the plurality of signals simultaneously. By this means, it is possible to shorten the time taken to form an image.

(Display Unit 110)

The display unit is an apparatus which displays image data output by a signal processing unit and typically employs a liquid crystal display, or the like. The display apparatus may also be provided separately from the photo acoustic image-forming apparatus according to the present invention.

(Holding Member 105 and Acoustic Matching Material 106)

The holding member is arranged in order to stabilize the shape of the object and the acoustic matching material is arranged in order to align the acoustic impedances of the holding member and the object. The acoustic impedance is a value which indicates the ease with which the acoustic wave passes through a material. At the boundary where the acoustic impedance changes, reflection and diffraction of the acoustic wave occurs. The greater the difference in acoustic impedance where the wave is reflected, the greater the energy intensity of the reflected wave, and the lower the energy intensity of the diffracted wave that is transmitted.

In order to receive an acoustic wave generated by an object with a probe, with as little reduction as possible in the energy intensity, generally, an acoustic matching material which gradually changes the acoustic impedance at the boundary is introduced. Therefore, when the acoustic impedance value of the acoustic matching material is compared with that of the materials on either side thereof, desirably, the acoustic impedance values show a uniform decrease or a uniform increase, with the acoustic impedance value of the acoustic matching material having a median value. Consequently, taking account of the fact that the acoustic impedance value of the holding member is generally larger than the object, it is desirable for the acoustic impedance value to become larger, in order, from the object, to the acoustic matching material, to the holding member.

The holding member or the acoustic matching material may adopt any form, provided that they satisfy the required functions, but a member having transmissive properties is used at least on the side where the light is irradiated. Typically, a poly-methyl platen or a plastic plate made of acrylic, or a glass plate, etc., is used. In general, these members are harder than a living organism and having a large acoustic impedance difference with respect to the living organism, and hence it is desirable to use an acoustic matching material.

As described above, in a composition in which the acoustic impedance becomes greater in order, from the object, to the acoustic matching material, to the holding member, the phase of the acoustic wave which is multiply reflected at the two boundary surfaces of the acoustic matching material is inverted through 180°. Furthermore, in cases where the acoustic wave generated by the object is reflected by the acoustic matching material, since the reflection occurs from a member having a high acoustic impedance to a member having a low acoustic impedance, the phase of the reflected wave is inverted through 180°. In this case also, it is possible to suppress phase inversion by means of the processing according to the present invention.

### First Embodiment

One example of a photoacoustic image-forming apparatus to which the present invention is applied will now be described. The apparatus used in shown in FIG. 1.

In the present embodiment, a YAG laser-pumped Ti:Sa laser system was used as the light source L. With this laser system, it is possible to irradiate light having a wavelength of 700 to 900 nm onto the object. The irradiation surface area of the laser light is enlarged by using an optical system, such as a mirror and a beam expander, and is then irradiated onto the object. The probe 104 employed was a probe with a two-dimensional configuration of 20 by 30 elements having an element width of 1 mm. In the front-stage processing unit 108, a 600 ch signal is received simultaneously from the probe, and subjected to amplification processing and digital conversion processing. The signal processing unit 109 processes this digital signal.

A human gastrocnemius muscle was measured as the object 101. A flat plate made from poly methyl pentene was used as the holding member 105. A gel-type acoustic matching member was used as the acoustic matching material 106.

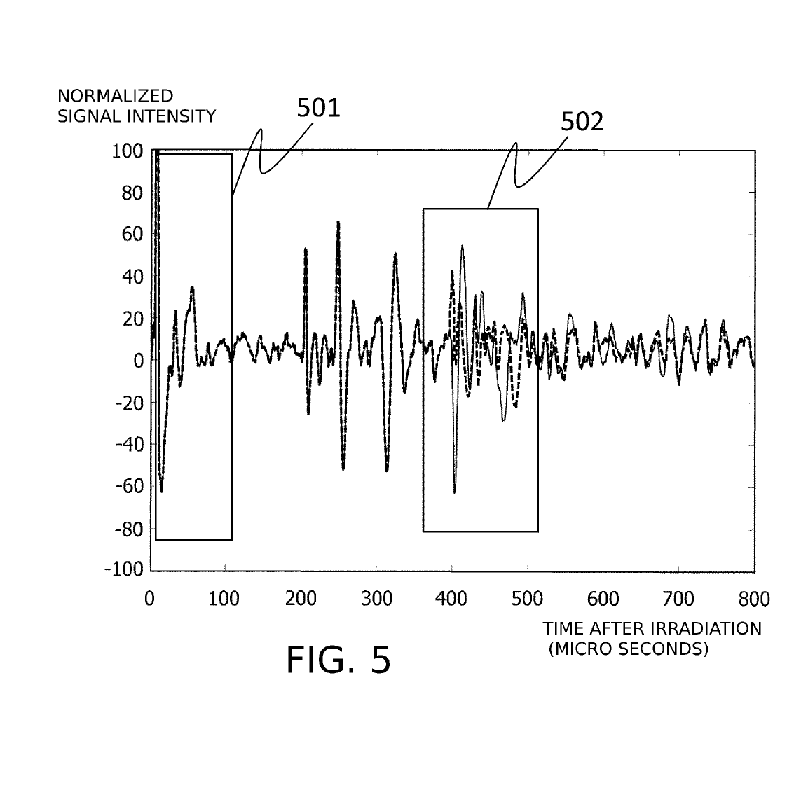

Next, the processing in the signal processing unit 109 will be described using FIG. 2 and FIG. 5. FIG. 5 is a graph which compares the acoustic wave signal obtained by a photoacoustic image-forming apparatus and a signal after suppression of reflected waves, in which the horizontal axis represents the time after irradiation of light (in microseconds) and the vertical axis represents the normalized signal intensity (the ratio with respect to the maximum amplitude).

The probe detected a photoacoustic wave generated by irradiating light having a wavelength of 800 nm onto the object, and a reflected wave of this photoacoustic wave, and the probe signal was passed through front-stage processing and input to the signal processing unit. The detection signal sampling unit 202 inside the signal processing unit sampled a sampling signal 501 respectively from each of the plurality of element signals (indicated by the frame on the left-hand side of FIG. 5). As the sampling conditions in this case, the signal width was set to 6 microseconds, which is the length of response of the probe, and the generation time was set to 10 microseconds. These sampling conditions were common for all of the detection signals. The average of the sampling signals was calculated to create a reference signal.

A correlation between the reference signal and the detection signal was calculated by the correlation calculation unit 205. From the calculated correlation results, a signal position having a large negative correlation value was taken to be a reflected signal position 502 (the frame on the right-hand side in FIG. 5). The width of the reflected signal was the same value of 6 microseconds as the reference signal. The reflected signal was predicted to appear with a delay of 20 to 40 microseconds from the sampling signal, and therefore the reflected signal position was selected to be within this predicted range.

A suppression signal was created by adjusting the intensity of the reference signal having an inverted phase, so as to match the maximum intensity of the reflected signal. The phase-inverted signal was suppressed by using this created suppression signal. FIG. 5 shows the reception signal (solid line) and the signal after suppression (dotted line). It can be seen that, at the reflected signal position 502, the reflected wave of the reception signal indicated by the solid line is diminished in the signal after suppression which is indicated by the dotted line.

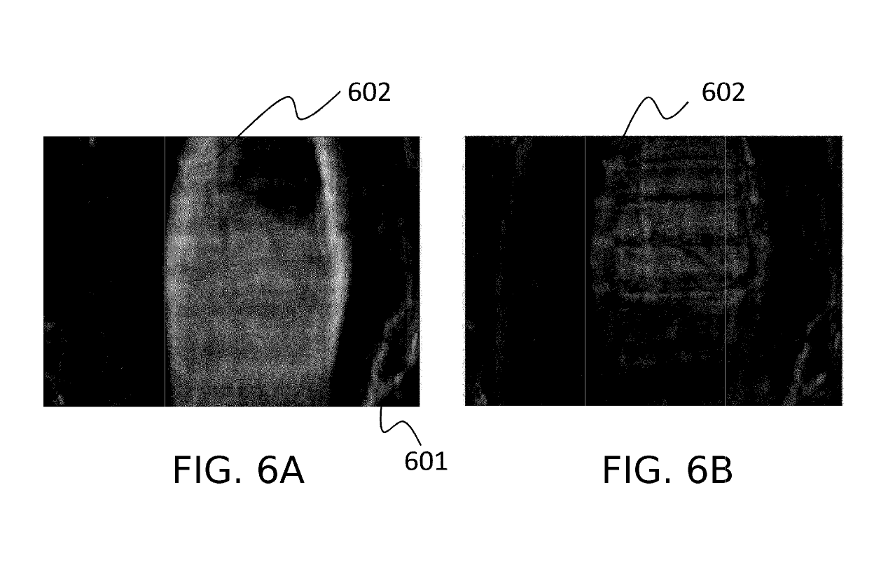

FIG. 6A is an image which has been reconstructed from a reception signal, without suppressing the reflected signal.

FIG. 6B is an image which has been reconstructed from a reception signal after suppression. The artifact image 602 produced by the reflected signal in FIG. 6A is suppressed in FIG. 6B, but the image of the absorbing body 601 is not suppressed. In this way, it is possible selectively to suppress an artifact caused by a reflected signal, by the signal processing according to the present invention.

### Second Embodiment

The basic composition of the apparatus according to this embodiment is the same as that of the first embodiment. However, the processing of the signal processing unit 109 is different to that of the first embodiment. In the first embodiment, the reference signal was created simply by averaging the sampling signal, but in the present embodiment, a reference signal was created by attenuating the frequency component through applying a filter to the averaged sampling signal.

The intensity of each frequency of the signal changes in direct proportion to exp (−ax). Here, a is the attenuation coefficient and x is the distance through which the signal has propagated. Therefore, the frequency intensity of the phase-inverted signal which is received by the probe after the sampling signal has been reflected and has propagated by an excess distance of δx is attenuated by exp (−aδx) with respect to the sampling data. This attenuated light is used as a filter and is multiplied by the sampling signal to give a reference signal.

In the composition of the present embodiment, the attenuation coefficient was 0.3 (dB/cm/MHz) and the distance of propagation due to reflection was 2 cm. After extending the number of sampling points of the sampling signal to a length equal to that of the detection signal, a Fourier transform was carried out to extract the frequency component, and an attenuation filter was applied. The correlation with the detection signal was calculated by using a signal that takes account of attenuation as the reference signal. From the correlation results, the phase-inverted signal position was identified and the phase-inverted signal was suppressed. As a result of this, it was possible to confirm that the intensity of the artifact due to the reflected signal having an inverted phase is diminished with respect to the first embodiment.

In the present embodiment, it is possible accurately to detect the phase-inverted signal by making the reference signal similar to the shape of the phase-inverted signal, which is a reflection component, and therefore the effect of suppressing the reflected signal can be improved.

### Third Embodiment

The basic composition of the apparatus according to this embodiment is the same as that of the first and second embodiments. In order to suppress the reflected signal of the photoacoustic signal generated by the object under investigation in the detection signal as disclosed in the present invention, it is necessary to extract the photoacoustic wave generated by the absorbing body under investigation, as a sampling signal. However, the detection signal also includes many waveforms other than that of the absorbing body under investigation. Therefore, in the present embodiment, in order to accurately sample the signal from the absorbing body under investigation in the detection signal, a signal waveform is added as a sampling condition.

The waveform which is added as a sampling condition uses a probe response waveform. The probe response is also called the impulse response, and is the waveform output by the probe when one pulse signal is input. A waveform which is similar to this probe response may be regarded as a signal originating in one photoacoustic signal. On the other hand, a signal in which a plurality of signals are intermixed has a signal waveform that is different to the probe response.

In the present embodiment, a shape correlation is created between a signal sampled from a detection signal, on the basis of sampling conditions, and a condition signal waveform which represents the probe response, and a waveform having a correlation no less than a prescribed threshold value is taken as the sampling waveform. Here, the position and width of the signal were used as sampling conditions, and the prescribed threshold value was set to 0.5. A reference signal was created by averaging a plurality of sampling waveforms obtained in this way.

Accordingly, by extracting a sampling signal that is similar to the shape of the probe response waveform, so as to create a reference signal, it is possible to make the reference signal waveform close to the shape of the reflected signal waveform. Consequently, it was possible to confirm that the intensity of the artifact due to the reflected signal having an inverted phase is diminished in comparison with the first embodiment.

In the present embodiment, by making the shape of the sampling signal close to the shape of the photoacoustic signal from the absorbing body under investigation, it is possible to accurately detect and suppress the phase-inverted signal.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-224945, filed on Oct. 10, 2012, which is hereby incorporated by reference herein in its entirety.

What is claimed is:

1. An object information acquiring apparatus, comprising:
a probe configured to convert an acoustic wave generated from an object irradiated with light into a detection signal; and
a signal processing unit configured to generate information about the interior of the object from the detection signal, wherein
the signal processing unit is configured to:
extract a sampling signal on the basis of signal sampling conditions, which are conditions relating to the signal shape, from the detection signal;
generate a reference signal from the sampling signal;
detect a phase-inverted signal, which is a portion of the detection signal where the phase is inverted with respect to the reference signal, by carrying out a correlation calculation of the detection signal and the reference signal;
generate a signal after suppression by suppressing the intensity of the phase-inverted signal from the detection signal; and
generate information about the interior of the object using the signal after suppression.

2. The object information acquiring apparatus, further comprising:
a holding member configured to hold the object via an acoustic matching material, wherein
the phase-inverted signal is generated from a reflected wave created due to the acoustic wave generated from the object being reflected at a boundary surface between the object and the acoustic matching member, or at a boundary surface between the acoustic matching material and the holding member.

3. The object information acquiring apparatus according to claim 2, wherein the reference signal has a shape of a phase-inverted signal originating in the reflected wave.

4. The object information acquiring apparatus according to claim 1, wherein the signal sampling conditions are determined so as to sample a signal corresponding to an absorbing body in the object, on the basis of the shape of the signal generated from the absorbing body.

5. The object information acquiring apparatus according to claim 4, wherein the signal sampling conditions include at least any one of an appearance time, width and intensity of the signal generated from the absorbing body.

6. The object information acquiring apparatus according to claim 1, wherein the reference signal is generated by averaging a plurality of sampling signals.

7. The object information acquiring apparatus according to claim 6, wherein the signal sampling conditions are changed for each sampling action of the plurality of sampling signals.

8. The object information acquiring apparatus according to claim 4, wherein the reference signal is generated by compensating for frequency attenuation in a signal generated from the absorbing body in the sampling signal.

9. The object information acquiring apparatus according to claim 4, wherein the signal sampling conditions are determined so as to compensate for change in the shape of signals due to response characteristics of the probe.

10. The object information acquiring apparatus according to claim 1, wherein the signal processing unit is configured to detect the phase-inverted signal by referring to the intensity of the detection signal.

11. The object information acquiring apparatus according to claim 1, wherein the signal processing unit is configured to use the reference signal to create a suppression signal for suppressing the intensity of the phase-inverted signal, and generate a signal after suppression by subtracting this suppression signal from the phase-inverted signal.

12. A control method for an object information acquiring apparatus including: a probe converting an acoustic wave generated from an object irradiated with light into a detection signal; and a signal processing unit generating information about the interior of the object from the detection signal,
the method comprising, by means of the signal processing unit:
extracting a sampling signal on the basis of signal sampling conditions, which are conditions relating to the signal shape, from the detection signal;
generating a reference signal from the sampling signal;
detecting a phase-inverted signal, which is a portion of the detection signal where the phase is inverted with respect to the reference signal, by carrying out a correlation calculation of the detection signal and the reference signal;
generating a signal after suppression by suppressing the intensity of the phase-inverted signal from the detection signal; and
generating information about the interior of the object using the signal after suppression.
